(19) United States
(12) Patent Application Publication
(10) Pub. No.: US 2016/0066892 A1
(43) Pub. Date: Mar. 10, 2016

(54) Cable Assembly for Converting a Consecutive Signaling Arrangement to an Interleaved Signaling Arrangement

(71) Applicant: Tyco Electronics Corporation, Berwyn, PA (US)

(72) Inventor: Edwin W. Schroder, West Lynn, OR (US)

(73) Assignee: Tyco Electronics Corporation, Berwyn, PA (US)

(21) Appl. No.: 14/482,560

(22) Filed: Sep. 10, 2014

(51) Int. Cl. A61B 8/00; H01B 13/06; A61B 8/08; H01B 7/00

(52) U.S. Cl. CPC A61B 8/56; H01B 7/009; H01B 13/06; A61B 8/4444; A61B 8/4483; A61B 8/52

(57) ABSTRACT

A cable assembly includes a first and second group of wires. Wires in the groups are arranged side-by-side. At respective first ends, the wires within the groups are held in place relative to one another so that the wires are separated by a first distance, and at respective second ends, the wires within the groups are held in place relative to one another so that the wires are separated by a second distance that is greater than the first distance. The first group of wires is overlaid on the second group of wires such that at first ends, the wires of the respective groups are consecutively arranged and at second ends, the wires of the respective groups are interleaved with one another.

### BACKGROUND

The present invention relates generally to a cabling arrangement for an ultrasound system. More specifically, the present invention relates to ribbon cable that facilitates coupling an ultrasound processor with a consecutive signaling arrangement to a probe that has an interleaved signaling arrangement.

Many medical devices include a base unit and a remote unit where the remote unit communicates information to and from the base unit. The base unit then processes information communicated from the remote unit and provides diagnostic information, reports, and the like. In some arrangements, a cable that includes a group of electrical wires couples the remote unit to the base unit. For example, signals may be communicated between an ultrasound probe and an ultrasound processor over a cable that includes hundreds of wires.

The wires within the cable may be provided in the form of a ribbon cable where conductors are arranged next to one another, side-by-side, and held together by a ribbonizing material, such as plastic. For ultrasound systems, the respective ends of the ribbon cable are connected to the ultrasound probe and the ultrasound processor. Connection of the probe and processor is relatively easy when the pin out arrangements on the probe and processor match.

When operating an ultrasound system in CW Doppler mode, the pins on the probe and processor may be configured so that half the pins are used for sending information to the transducers of the probe and the other half of the pins are used for receiving information from the transducers within the probe. In some ultrasound systems, this is accomplished by arranged the pins on the processor and probe so that a first group of adjacent pins is used to transmit transducer signals and a second group of adjacent pins is used to receive transducer signals. For example, pins 1-8 on both the probe and the processor may be used for sending transducer information, and pins 9-16 may be used for receiving transducer information.

However, in other systems, the pin out arrangement of the probe and the processor do not match. For example, instead of having the grouped arrangement described above, the pin out on the probe may be configured so that the function (i.e., receiving and sending) alternates between odd and even-numbered pins. For example, pins one and three on the probe may be used for sending signals to transducers numbered one and two of the probe. Pins two and four may be used for receiving signals from the transducers numbered one and two, and so on. When the pin outs on the probe and processor do not match, an operator typically has to peel individual wires out the ribbon at one end or the other and re-arrange the wires so that the signals are communicated correctly.

### SUMMARY

In one aspect, a cable assembly includes a first group of wires arranged side-by-side. At a first end, the wires are held in place relative to one another so that the wires are separated by a first distance; and at a second end, the wires are held in place relative to one another so that the wires are separated by a second distance that is greater than the first distance. The cable includes a second group of wires arranged side-by-side. At a first end, the second group of wires are held in place relative to one another so that they are separated by a third distance; and at a second end, the wires are held in place relative to one another so that they are separated by a fourth distance that is greater than the third distance. The first group of wires is overlaid on the second group of wires such that the first end of the first group of wires is adjacent to the first end of the second group of wires, and each wire of the second end of the first group of wires is interleaved with a wire of the second end of the second group of wires.

In a second aspect, a method for manufacturing a cable includes arranging a first group of wires side-by-side so that at a first end, the wires are separated by a first distance; and at a second end, the wires are separated by a second distance that is greater than the first distance. A ribbonizing material is applied over the first end and the second end of the first group of wires to hold the wires in place. The method also includes arranging a second group of wires side-by-side so that at a first end, the wires are separated by a third distance; and at a second end, the wires are separated by a fourth distance that is greater than the third distance. A ribbonizing material is applied over the first end and the second end of the second group of wires to hold the wires in place. The first group of wires is overlaid on the second group of wires such that the first end of the first group of wires is adjacent the first end of the second group of wires, and each wire of the second end of the first group of wires is interleaved with a wire of the second end of the second group of wires.

In a third aspect, an ultrasound system includes an ultrasound probe, an ultrasound processor, and a cable assembly for communicating signals between the ultrasound probe and the ultrasound processor. The ultrasound probe has a first group of electrical contacts for sending transducer signals, and a second group of electrical contacts interleaved with the first group of electrical contacts for receiving transducer signals. The ultrasound processor has a first group of electrical contacts for sending transducer signals, and a second group of electrical contacts adjacent to the first group of electrical contacts for receiving transducer signals. The cable includes a first group of wires arranged side-by-side. At a first end, the wires are held in place relative to one another so that the wires are separated by a first distance; and at a second end, the wires are held in place relative to one another so that the wires are separated by a second distance that is greater than the first distance. The cable includes a second group of wires arranged side-by-side. At a first end, the second group of wires are held in place relative to one another so that they are separated by a third distance; and at a second end, the wires are held in place relative to one another so that they are separated by a fourth distance that is greater than the third distance. The first group of wires is overlaid on the second group of wires such that the first end of the first group of wires is adjacent to the first end of the second group of wires, and each wire of the second end of the first group of wires is interleaved with a wire of the second end of the second group of wires.

### BRIEF DESCRIPTION OF THE DRAWINGS

FIG. 1 illustrates an ultrasound system that includes a probe with an interleaved signaling arrangement and a processor with a consecutive signaling arrangement.

FIG. 2 illustrates first and second groups of wires.

FIGS. 3A and 3B illustrate assembly of the first and second groups of wires into a cable assembly.

FIG. 4 illustrates a top view of the cable assembly.

### DETAILED DESCRIPTION

A cable assembly that overcomes the problems above is disclosed in detail below. Generally, the cable assembly includes a first group of wires and a second group of wires. At first ends of the respective groups of wires, the wires are spaced apart by a first distance. At second ends of the respective groups of wires, the wires are spaced apart by a second distance that is about double the first distance. The second group of wires is overlaid on top of the second group of wires so that, at the second ends, the respective wires of the groups are interleaved, while at the first end, the respective wires of the groups are arranged in a consecutive arrangement.

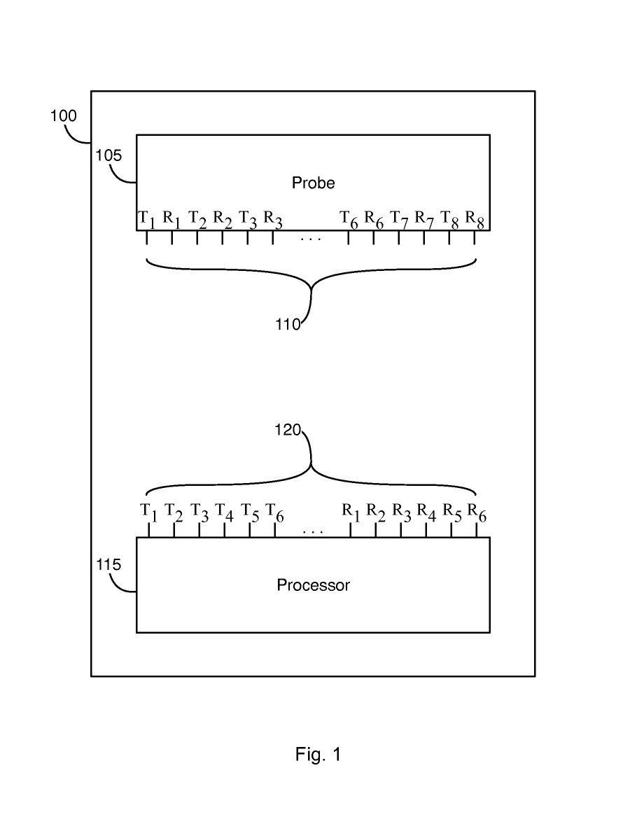

FIG. 1 illustrates an exemplary ultrasound system 100 that may include an ultrasound processor 115 and a probe 110. The probe 110 may include a number of transducers (not shown) configured to transmit an ultrasonic wave and to receive a corresponding echo of the ultrasonic wave. The probe 110 also includes a group of pins 110 arranged in an odd/even or interleaved relationship, where odd-numbered pins are used to transmit signals to respective transducers, and even-numbered pins are used to receive signals from respective transducers. Each transducer is operatively coupled to a pair of pins 110 on the probe. For example, a first transducer is coupled to pins T₁ and R₁, a second transducer is coupled to pins T₂ and R₂, and so on. While only a few pins are illustrated, it should be understood that, in practice, the number of pins might be much larger.

The processor 115 may correspond to any system capable of communicating transducer signals to and from a probe 105 for generating images associated with transducer signals. The processor 120 includes a group of pins 120 configured to send and receive information to corresponding pins 110 of the probe. As illustrated, the pins 120 may be arranged so that all the pins for transmitting transducer signals are grouped together on one side, and all the pins for receiving transducer signals from the probe are grouped together on the other side. In operation, pins 120 on the processor 115 are wired or coupled to like-named pins 110 on the probe. For example, pin T1 on the processor 120 is wired or coupled to pin T1 on the probe 105. The number of pins shown is only exemplary and, in practice, might be much larger.

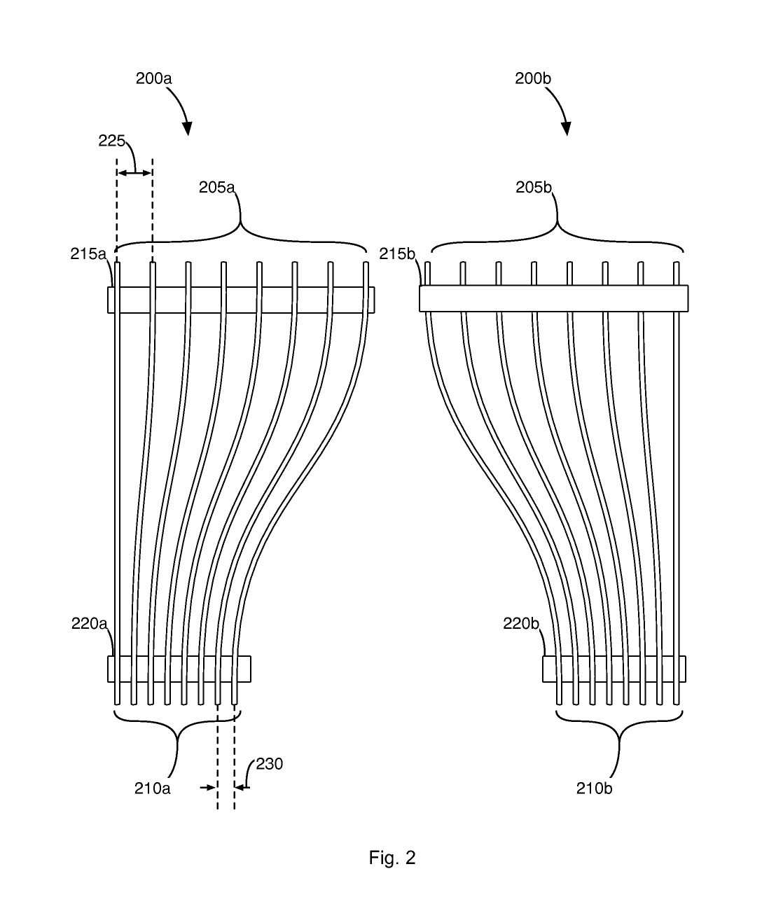

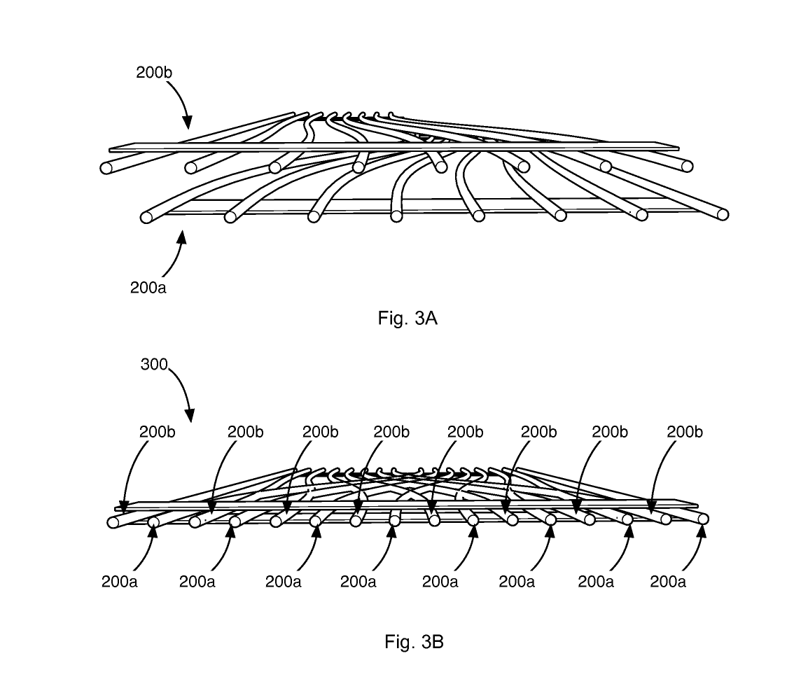

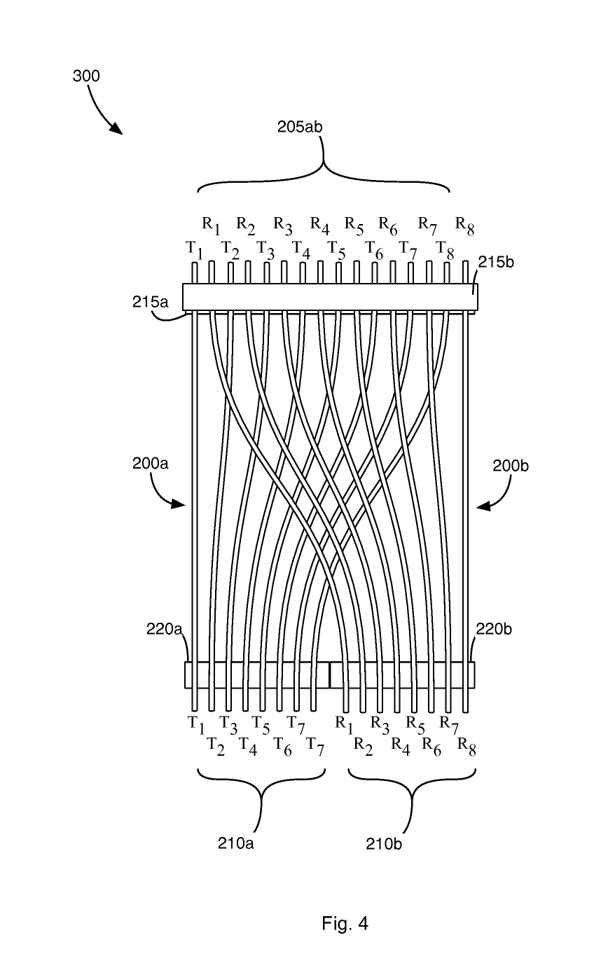

FIG. 2 illustrates portions of an exemplary cable assembly 300 (See FIGS. 3B and 4) for coupling the pins 120 of the processor 115 to the pins 110 of the probe 105. The cable assembly 300 includes a first group of wires 200a arranged side-by-side, and a second group of wires 200b arranged side-by-side. In an exemplary implementation, each wire may correspond to a coax wire that includes at least a center conductor and a shield. For example, the coax wire may be a micro coax wire that has diameter of about 0.014 inches (0.03556 cm). A different wire, which has a different number of conductors and/or configuration, may be utilized.

At the first end 210a, the wires of the first group of wires 200a may be spaced apart by a distance 230 that may correspond roughly to the diameter of each wire so that adjacent wires touch one another. For example, the wires may be spaced apart by about 0.017 inches (0.04318 cm). The wires may be spaced apart by a different distance. At the second end 205a, the wires may be spaced apart by a distance 225 that is greater than the first distance 230. For example, the distance 225 between the wires at the second end 205 may correspond to roughly 2× the diameter of each wire, or about 0.028 inches (0.07112 cm). The spacing between the wires at the first end 210b and the second end 205b of the second group of wires 200b may be similarly configured.

In some implementations, a ribbonizing material 220ab may be applied near respective first ends 210ab of the first and second groups of wires 200ab to hold the wires at the first ends 210ab together. A ribbonizing material 215ab may also be applied near respective second ends 205ab of the first and second groups of wires 200ab to hold the wires at the second ends 205ab together. The ribbonizing material 215ab and 220ab may correspond to an adhesive-backed material, such as polyester film, that may have with a width of between about 1 inch (2.54 cm) and 3 inches (7.62 cm). In alternative implementations, the ribbonizing material may extend the length of the wires 200ab. For example, the ribbonizing material may be applied to the wires 200ab during an extrusion process or other manufacturing processes for ribbonizing a group of wires.

The ribbonizing material 215a at the second end 205a of the first group of wires 200a may be disposed along a bottom side of the group of wires 200a, and the ribbonizing material 215b at the second end 205b of the second group of wires 200b may be disposed along a top side of the group of wires 200b, as illustrated in FIG. 2. The opposite arrangement is also possible. The ribbonizing material 220ab at the respective first ends 210ab of the first and second groups of wires 200ab may be disposed on either side of the respective groups of wires 200ab.

In operation, the first and second groups of wires 200ab are arranged in the manner described above. As illustrated in FIG. 3A, the second group of wires 200b may be brought over the first group of wires 200a. Next the second group of wires 200b is lowered on to the first group of wires 200a so the ends of the wires are substantially aligned with one another, as illustrated in FIG. 3B, to provide the assembled cable 300. In some implementations, the ribbonizing material 215ab secures the second ends 205ab of the first and second groups of wires 200ab in this configuration. For example, an adhesive on the ribbonizing material 215ab may secure the second ends 205ab in this configuration.

As can be seen in FIG. 4, the cable assembly 300 overcomes the problems described above by converting a consecutive signaling arrangement to an odd/even or interleaved signaling arrangement. For example, the first end 210a of the first group of wires 200a may be coupled to the transmit pins of the ultrasound processor 125 described above, and the first end 210b of the second group of wires 200b may be coupled to the receive pins. The interleaved second ends 205ab of the first and second groups of wires 200ab may be coupled to the interleaved transmit and receive pins of the ultrasound probe 105. This arrangement advantageously does away with the step of peeling apart wires, which is required in known cables.

While the cable assembly 300 has been described with reference to certain embodiments, it will be understood by those skilled in the art that various changes may be made and equivalents may be substituted without departing from the spirit and scope of the claims of the application. Various modifications may be made to adapt a particular situation or material to the teachings disclosed above without departing from the scope of the claims. Therefore, the claims should not be construed as being limited to any one of the particular embodiments disclosed, but to any embodiments that fall within the scope of the claims.

What is claimed is:

1. A cable assembly comprising:
a first plurality of wires arranged side-by-side, wherein at a first end, the wires are held in place relative to one another so that at the first end, the wires are separated by a first distance, and at a second end, the wires are held in place relative to one another so that at the second end, the wires are separated by a second distance that is greater than the first distance;
a second plurality of wires arranged side-by-side, wherein at a first end, the second plurality of wires are held in place relative to one another so that at the first end, the wires are separated by a third distance, and at a second end, the second plurality of wires are held in place relative to one another so that at the second end, the wires are separated by a fourth distance that is greater than the third distance,
wherein the first plurality of wires is overlaid on the second plurality of wires such that the first end of the first plurality of wires is adjacent to the first end of the second plurality of wires, and each wire of the second end of the first plurality of wires is interleaved with a wire of the second end of the second plurality of wires.

2. The cable according to claim 1, wherein the second distance between the wires of the second end of the first plurality of wires is greater than or equal to the diameter of a wire of the second plurality of wires.

3. The cable according to claim 1, wherein the first plurality of wires are held in place at the second end via a ribbonizing material that runs along a bottom of the first plurality of wires, and the second plurality of wires are held in place at the second end via a ribbonizing material that runs along a top of the first plurality of wires.

4. The cable according to claim 3, wherein the first plurality of wires are held in place at the first end via a ribbonizing material that runs along either a top or a bottom of the first plurality of wires, and the second plurality of wires are held in place at the first end via a ribbonizing material that runs along either a top or a bottom of the first plurality of wires.

5. The cable according to claim 3, wherein the ribbonizing material corresponds to an adhesive-backed material.

6. The cable according to claim 1, wherein the first end of the first plurality of wires is configured to be coupled to the first group of electrical contacts for sending transducer signals of the ultrasound processor, and the first end of the second plurality of wires is configured to be coupled to the second group of electrical contacts for receiving transducer signals of the ultrasound processor.

7. The cable according to claim 6, wherein the second end of the first plurality of wires is configured to be coupled to the first group of electrical contacts for receiving transducer signals of the ultrasound probe, and the second end of the second plurality of wires is configured to be coupled to the second group of electrical contacts for sending transducer signals of the ultrasound processor.

8. The cable according to claim 1, wherein each wire of the first and second plurality of wires corresponds to a coax wire that includes at least a center conductor and a shield.

9. A method for manufacturing a cable comprising:
arranging a first plurality of wires side-by-side so that at a first end, the wires are separated by a first distance, and at a second end, the wires are separated by a second distance that is greater than the first distance;
applying a ribbonizing material over the first end and the second end of the first plurality of wires to hold the wires in place;
arranging a second plurality of wires side-by-side so that at a first end, the wires are separated by a third distance, and at a second end, the wires are separated by a fourth distance that is greater than the third distance;
applying a ribbonizing material over the first end and the second end of the second plurality of wires to hold the wires in place;
overlaying the first plurality of wires on the second plurality of wires such that the first end of the first plurality of wires is adjacent to the first end of the second plurality of wires, and each wire of the second end of the first plurality of wires is interleaved with a wire of the second end of the second plurality of wires.

10. The method according to claim 9, wherein the second distance between the wires of the second end of the first plurality of wires is greater than the diameter of a wire of the second plurality of wires.

11. The method according to claim 9, wherein the ribbonizing material that holds the first plurality of wires in place at the second end runs along a bottom of the first plurality of wires, and the ribbonizing material that holds the second plurality of wires in place at the second end runs along a top of the first plurality of wires.

12. The method according to claim 11, wherein the ribbonizing material that holds the first plurality of wires in place at the first end runs along either a top or a bottom of the first plurality of wires, and the ribbonizing material that holds the second plurality of wires in place at the first end runs along either a top or a bottom of the first plurality of wires.

13. The method according to claim 11, wherein the ribbonizing material corresponds to an adhesive-backed material.

14. The method according to claim 9, further comprising coupling the first end of the first plurality of wires to the first group of electrical contacts for sending transducer signals of the ultrasound processor, and the first end of the second plurality of wires to the second group of electrical contacts for receiving transducer signals of the ultrasound processor.

15. The method according to claim 14, further comprising coupling the second end of the first plurality of wires to the first group of electrical contacts for receiving transducer signals of the ultrasound probe, and coupling the second end of the second plurality of wires to the second group of electrical contacts for sending transducer signals of the ultrasound processor.

16. The method according to claim 9, wherein each wire of the first and second plurality of wires corresponds to a coax wire that includes at least a center conductor and a shield.

17. An ultrasound system comprising:
an ultrasound probe having a first group of electrical contacts for sending transducer signals, and a second group of electrical contacts interleaved with the first group of electrical contacts for receiving transducer signals;
an ultrasound processor having a first group of electrical contacts for sending transducer signals, and a second group of electrical contacts adjacent to the first group of electrical contacts for receiving transducer signals; and
a cable assembly for communicating signals between the ultrasound probe and the ultrasound processor, the cable assembly comprising:
a first plurality of wires arranged side-by-side, wherein at a first end, the wires are held in place relative to one another so that at the first end, the wires are separated by a first distance, and at a second end, the wires are held in place relative to one another so that at the second end, the wires are separated by a second distance that is greater than the first distance;
a second plurality of wires arranged side-by-side, wherein at a first end, the second plurality of wires are held in place relative to one another so that at the first end, the wires are separated by a third distance, and at a second end, the second plurality of wires are held in place relative to one another so that at the second end, the wires are separated by a fourth distance that is greater than the third distance,
wherein the first plurality of wires is overlaid on the second plurality of wires such that the first end of the first plurality of wires is adjacent to the first end of the second plurality of wires, and each wire of the second end of the first plurality of wires is interleaved with a wire of the second end of the second plurality of wires.

18. The ultrasound system according to claim 17, wherein the second distance between the wires of the second end of the first plurality of wires is greater than the diameter of a wire of the second plurality of wires.

19. The ultrasound system according to claim 17, wherein the first plurality of wires are held in place at the second end via a ribbonizing material that runs along a bottom of the first plurality of wires, and the second plurality of wires are held in place at the second end via a ribbonizing material that runs along a top of the first plurality of wires.

20. The ultrasound system according to claim 19, wherein the first plurality of wires are held in place at the first end via a ribbonizing material that runs along either a top or a bottom of the first plurality of wires, and the second plurality of wires are held in place at the first end via a ribbonizing material that runs along either a top or a bottom of the first plurality of wires.
